(19) United States
(12) Patent Application Publication
(10) Pub. No.: US 2001/0031273 A1
(43) Pub. Date: Oct. 18, 2001

(54) Use of a dispersion based on lipid vesicles as an anti-inflammatory composition

(75) Inventor: Jean-Thierry Simonnet, Paris (FR)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: L'OREAL, Paris (FR)

(21) Appl. No.: 09/859,536

(22) Filed: May 18, 2001

Related U.S. Application Data
(63) Continuation of application No. 09/141,333, filed on Aug. 27, 1998.

(30) Foreign Application Priority Data
Aug. 27, 1997 (FR) 9710709

(51) Int. Cl. A61K 9/127; A61K 31/198; A61K 31/201; A61K 31/20

(52) U.S. Cl. 424/401; 514/558; 514/564; 514/565

(57) ABSTRACT

The present invention relates to the use of an aqueous dispersion of oily globules which have a monolamellar or oligolamellar liquid crystal coating, in a cosmetic composition, or for the preparation of a dermatological composition, for preventing and/or combating skin disorders involving an inflammatory process, in particular for preventing and/or treating skin irritation and/or for preventing and/or treating solar erythema.

### BACKGROUND OF THE INVENTION

1. Field of the Invention

The present invention relates to the use of an aqueous dispersion of oily globules, each provided with a monolamellar or oligolamellar liquid crystal coating, in a cosmetic composition, or for the preparation of a dermatological composition, for preventing and/or combating skin disorders involving an inflammatory process, in particular for preventing and/or treating skin irritation and/or for preventing and/or treating solar erythema.

The invention also relates to a process for the cosmetic treatment of inflammation, irritation and/or solar erythema by applying such a dispersion.

2. Description of the Related Art

Inflammation (or the inflammatory process) is a set of biological reactions found throughout the animal kingdom. In man, two out of every three patients exhibit an inflammatory syndrome. Inflammation can be localized. It can be defined as the first response to any local attack by a series of non-specific reactions which are triggered irrespective of the initial cause, and taking place in three stages: vascular, cellulovascular and tissue fibrosis.

Localized inflammation, and in particular skin inflammation, is characterized by symptoms such as swelling, pain, redness and heat.

Skin inflammation phenomena include, for example, erythemas, in particular those caused by ultraviolet and sunlight, pruritus, erythema nodosum, urticaria, systemic mastocytosis, psoriasis, insect bites, other dermatological complaints such as atrophic polyclhondritis, erythemalgia, lipoid necrobiosis, disseminated erythemal lupus or any skin irritation caused by contact with corrosive products such as certain household products.

Substances for preventing or treating inflammation have been sought for many years in the pharmaceutical and cosmetics industry. In this respect, many substances have already been described in the literature under the names steroidal or non-steroidal anti-inflammatory agents (SAI or NSAI), a description of which will be found, for example, in the book by Schorderet and Dayer “Pharmacologie, des concepts fondamentaux aux applications therapeutiques”, 1992, chapter 37, pages 541-561, 2nd edition, published by Frison-Roche/Slatkine.

In view of the fact that the known anti-inflammatory agents often have appreciable side effects, it is apparent that there exists a need to provide novel products with anti-inflammatory activity.

### SUMMARY OF THE INVENTION

One object of the present invention is thus to provide a cosmetic and/or dermatological product which has anti-inflammatory activity while at the same time having no appreciable side effects.

The Applicant has found, surprisingly, that a dispersion based on oily globules coated with a larnellar, and more particularly a monolamellar or oligolamellar, liquid crystal phase has anti-inflammatory properties and can prevent an inflammatory reaction. The dispersion according to the invention can be used in particular to prevent and/or treat solar erythema. In addition, the dispersion according to the invention can be used when the inflammatory reaction is caused by a skin irritation induced by contact with an irritant product.

Thus, an object of the present invention is to provide a method for preventing and/or combating skin disorders involving an inflammatory process and/or for preventing and/or treating skin irritation and/or for preventing and/or treating solar erythema by applying to the skin a cosmetic composition including an aqueous dispersion of oily globules, each provided with a monolamellar or oligolamellar liquid crystal coating,.

Another object of the present invention is to provide a process for the treatment of inflammation and/or skin irritation and/or solar erythema, which includes applying an aqueous dispersion of oily globules, each provided with a monolamellar or oligolamellar liquid crystal coating, to inflamed and/or irritated and/or erythemal skin.

With the foregoing and other objects, advantages and features of the invention that will become hereinafter apparent, the nature of the invention may be more clearly understood by reference to the following detailed description of the preferred embodiments of the invention and to the appended claims.

### DETAILED DESCRIPTION

The cosmetic or dermatological composition containing the dispersion according to the invention includes a physiologically acceptable medium, i.e., one which is compatible with the skin, the scalp and the hair.

The oily globules of the invention include a mono- or oligolamellar membrane of ionic or nonionic lipids containing an oily phase (lipid vesicles with an oily core). The term oligolamellar layer is understood to refer to a layer comprising from 2 to 5 lipid lamellae. The average size of the coated oily globules is less than 500 manometers and, for example, is about 200 manometers. Due to their small size, penetration of the globules into the intercormeocytic spaces, which are of comparable size, is greatly facilitated.

The dispersion according to the invention is of the emulsion type, in particular an oil-in-eater (O/W) type emulsion, containing the oily globules provided with a lamellar liquid crystal coating. In particular, these emulsions are of the type described in European Patent Applications EP-A-0,641,557 and EP-A-0,705,593, which are incorporated herein by reference in their entireties.

Thus, according to a first preferred embodiment of the invention, the oily globules are of the type described in patent application EP-A-0,641,557 (corresponding to U.S. Pat. No. 5,658,575). In accordance with this application, each oily globule is individually coated with a monolamellar or oligolamellar layer obtained from at least one lipophilic surfactant, from at least one hydrophilic surfactant and from at least one fatty acid, and the aqueous phase of the emulsion according to the invention contains a dissolved basic agent.

The lipophilic surfactant can be chosen from the group comprising sucrose distearate, diglyceryl distearate, tetraglyceryl tristearate, decaglyceryl decastearate, diglyceryl monostearate, hexaglyceryl tristearate, decaglyceryl pentastearate, sorbitan monostearate, sorbitan tristearate, diethylene glycol monostearate, the glyceryl ester of palmitic and stearic acids, the monostearate polyoxyethylenated with 2 EO (comprising 2 ethylene oxide units), glyceryl mono- and dibehenate, and pentaerythritol tetrastearate. Preferably, the lipophilic surfactant is sucrose distearate.

The lipophilic surfactant preferably has an HLB (hydrophilic-lipophilic balance) ranging from 2 to 5.

The hydrophilic surfactant can be chosen from the group comprising sorbitan monostearate polyoxyethylenated with 4 mol of ethylene oxide (Polysorbate 61), sorbitan tristearate polyoxyethylenated with 20 mol of ethylene oxide, the monostearate polyoxyethylenated with 8 mol of ethylene oxide, hexaglyceryl monostearate, the monostearate polyoxyethylenated with 10 mol of ethylene oxide, the distearate polyoxyethylenated with 12 mol of ethylene oxide and methylglucose distearate polyoxyethylenated with 20 mol of ethylene oxide. Preferably, the hydrophilic surfactant is Polysorbate 61.

The hydrophilic surfactant preferably has an HLB ranging from 8 to 12.

According to one embodiment of the invention, the lipophilic surfactant and the hydrophilic surfactant contain at least one saturated fatty chain having more than 12 carbon atoms, preferably ranging from 16 to 22 carbon atoms.

According to one embodiment of the invention, the fatty acid contains at least one saturated fatty chain having more than 12 carbon atoms, preferably ranging from 16 to 22 carbon atoms. Preferably, the fatty acid is chosen from the group comprising palmitic acid, stearic acid, arachidic acid and behenic acid. Even more preferably, the fatty acid is stearic acid.

The basic agent can be chosen from inorganic bases and organic bases, in particular amphoteric bases, i.e., bases having both anionic and cationic functional groups. Examples of possible basic agents are sodium hydroxide, triethanolamine, lysine and arginine. The basic agent is preferably lysine.

The basic agent is dissolved in the aqueous phase in an amount at least equal to the amount required to neutralize the fatty acid.

According to a second preferred embodiment of the invention, the oily globules are of the type described in patent application EP-A-0,705,593. In accordance with this application, the oily globules are individually coated with a monolamellar or oligolamellar layer obtained from at least one lipophilic surfactant and from at least one hydrophilic surfactant as are defined above, and also from at least one ionic amphiphilic lipid which gives the composition a pH ranging from 5.5 to 7.5.

The ionic amphiphilic lipid used in the context of the present invention is preferably chosen from the group comprising neutralized anionic lipids, amphoteric lipids and alkylsulphonic derivatives.

The neutralized anionic lipids are chosen in particular from:

alkali metal salts of dicetyl phosphate, and in particular the sodium and potassium salts;

alkali metal salts of dimyristyl phosphate, and in particular the sodium and potassium salts;

alkali metal salts of cholesteryl sulphate, and in particular the sodium salt;

alkali metal salts of cholesteryl phosphate, and in particular the sodium salt;

the monosodium and disodium salts of acylglutamic acids, and in particular the monosodium and disodium salts of N-stearoylglutamic acid;

the sodium salt of phosphatidic acid.

The amphoteric lipids are chosen in particular from phospholipids, and in particular from pure soybean phosphatidyl ethanolamine.

The alkylsulphonic derivatives are advantageously the compounds of formula:

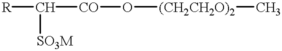

in which R represents the radicals C₁₆H₃₃ and C₁₈H₃₇ taken as a mixture or separately, and M is an alkali metal, preferably sodium.

Preferably, the ionic amphiphilic lipid is the monosodium salt of N-stearoylglutamic acid sold under the trade name “Amisofit HS 11” by Ajinomoto, or alternatively the disodium salt of N-stearoylglutamic acid sold under the trade name “Amisoft HS 21” by Ajinomoto.

The coating according to the second embodiment of the invention, of the oily globules, is preferably obtained using a total amount of hydrophilic surfactant, of lipophilic surfactant and of ionic amphiphilic lipid of between about 2% and about 6% by weight relative to the total weight of the composition. Even more preferably, this amount is between 3% and 4%. The relative amounts of lipophilic and hydrophilic surfactants and of ionic amphiphilic lipid preferably vary within the following respective ranges: 35-55% /25-40% /15-35% by weight relative to their total weight.

The fatty phase, and in particular that of the coated oily globules, preferably represents 5 to 50% by weight relative to the total weight of the composition. Even more preferably, this percentage is between 10 and 40%. Preferably, the oil/water ratio is equal to or less than 1.

The weight ratio of the oily globules to the elements constituting the coating is preferably from 3 to 13, more preferably from 5 to 8; even more preferably, this ratio is equal to about 7.

The oils which can be used in the fatty phase are the oils used conventionally in cosmetic or dermatological compositions, such as, for example, animal oils, plant oils (sunflower oil), mineral oils, synthetic oils, silicone oils (cyclomethicone), fluoro oils and perfluoro oils. The fatty phase can also comprise fatty alcohols, fatty acid esters and waxes (hydrogenated milk wax).

The dispersions according to the invention can also contain, in the oily globules, a fatty or lipophilic substance chosen from antioxidants, anti-free-radical agents, melanoregulators, tanning accelerators, depigmenting agents, skin-colouring agents, liporegulators, slimming agents, anti-acne agents, antiseborrhoeic agents, anti-aging agents, anti-wrinkle agents, anti-UV agents, keratolytic agents, anti-inflammatory agents, refreshing agents, cicatrizing agents, vascular protection agents, antibacterial agents, antifungal agents, antiperspirants, deodorants, hair conditioners, immunomodulators, nourishing agents, essential oils and fragrances.

As examples of lipophilic active agents for treating the skin and/or the hair, which can be used in the context of the present invention, mention may be made of the following compounds:

D-α-tocopherol, DL-α-tocopherol, D-α-tocopheryl acetate, DL-α-tocopheryl acetate, ascorbyl palmitate, vitamin F glycerides, D vitamins and in particular vitamin D₂ and vitamin D₃, retinol, retinol esters (retinyl palmitate, retinyl propionate), β-carotene, D-panthenol, farnesol, farnesyl acetate, oils rich in essential fatty acids and in particular jojoba oil and blackcurrant oil, 5-n-octanoylsalicylic acid, salicylic acid, alkyl esters of α-hydroxy acids such as citric acid, lactic acid and glycolic acid, Asiatic acid, madecassic acid, asiaticoside, total extract of Centella asiatica, β-glycyrrhetinic acid, a-bisabolol, ceramides and in particular 2-oleoylamino-1,3octadecane, phytanetriol, milk sphingomyelin, phospholipids of marine origin rich in polyunsaturated essential fatty acids, ethoxyquin, extract of rosemary, extract of balm, quercitin, extract of dried microalgae (Algoxan red sold by Algatec), essential oil of bergamot, octyl methoxycinnamate (Parsol MCX sold by Givaudan-Roure), butylmethoxydibenzoylmethane (Parsol 1789 sold by Givaudan-Roure), octyl triazone (Uvinul T150 sold by BASF), yellow, brown, black and red iron oxides, titanium oxides, which can be in micrometric or nanometric form or in coated form (for example coated with a perfluoroalkyl), 3,5-di-tert-butyl-4-hydroxy-3benzylidenecamphor, 2-benzotriazol-2-yl-4-methyl-6-[3 -[1,3,3,3 -tetramethyl-1-[(trimethylsilyl)oxyl]-disiloxanyl]-2-methylpropyl]phenol, perfluoro oil (perfluorodecalin, perfluorooctyl bromide), hyperoxygenated corn oil (Epaline 100 sold by the company Carilene).

The compositions of the invention can also comprise adjuvants that are conventional in the cosmetics or dermatological field, chosen in particular from organic solvents, ionic or nonionic thickeners, softeners, antioxidants, opacifiers, stabilizers, emollients, antifoaming agents, fragrances, preserving agents, surfactants, fillers, sequestering agents (EDTA), polymers, propellants, basifying or acidifying agents, dyes, hydrophilic or lipophilic active agents or any other ingredient usually used in cosmetics or in the dermatological field. The amounts of these various adjuvants are those used conventionally in the fields considered, and, for example, from 0.01 to 20% of the total weight of the composition. As can be appreciated by one of ordinary skill in the art, these adjuvants must be of a nature and used in an amount such that they do not disrupt the dispersion of the invention.

The organic solvents may include, but are not limited to lower polyols and alcohols.

The thickeners can be chosen in particular from carboxyvinyl polymers, and modified or unmodified guar gums and cellulose gums, such as, for example, hydroxypropyl guar gum, xanthan gum, methylhydroxyethyl-cellulose, hydroxypropylmethylcellulose or hydroxy-ethylcellulose.

These compositions can in particular comprise one or more hydrophilic or lipophilic, cosmetically or dermatologically active compounds which are free or encapsulated in vesicles, chosen, for example, from moisturizers (glycerol, propylene glycol, hydroxyproline), vitamins and keratolytic agents (hydroxy acids).

The compositions of the invention can be used, for example, as anti-sun or after-sun compositions on the human epidermis or on the hair. They can be in the form of creams, milks or fluid lotions.

Having generally described this invention, a further understanding can be obtained by reference to certain specific examples which are provided herein for purposes of illustration only and are not intended to be limiting unless otherwise specified.

### EXAMPLES

### Example 1

### Soothing Composition

| Phase A | | |
|---|---|---|
| Sucrose distearate sold by the company | | 2% |
| “Stearinerie Dubois” | | |
| Polysorbate 61 sold under the name | | 1.3% |
| “Tween 61” by the company ICI | | |
| Stearic acid | | 1% |
| Stearyl heptanoate/stearyl octanoate | | 5% |
| Hydrogenated milk wax sold by Ichimaru | | 3.2% |
| Pharco under the name Milkwhity | | |
| Farnesol/farnesyl acetate (mixture sold | | 3% |
| by Induchem under the name Unibiovit B33) | | |
| Cyclomethicone | | 3.7% |
| Sunflower oil | | 10% |
| D-α-tocopheryl acetate | | 1% |
| Preserving agent | | 0.15% |
| Phase B: | | |
| Glycerol | | 3% |
| EDTA | | 0.05% |
| Hydroxyproline | | 1% |
| Lysine | | 0.5 |
| Propylene glycol | | 0.7% |
| Preserving agent | | 0.4% |
| Demineralized water | | 55% |
| Phase C: | | |
| Silicone gum sold by Dow Corning under | | 4% |
| the name “Q2-1403 Fluid” | | |
| Phase D: | | |
| Carboxyvinyl polymer sold by the company | | 0.1% |
| Sigma under the name “Synthalen K” | | |
| Demineralized water | qs | 100% |

The manufacturing process includes the steps of bringing phase A and phase B to 75° C., rapidly introducing phase B into phase A with vigorous stirring and maintaining the temperature of 75° C. and the stirring for 15 to 30 minutes. The mixture is then subjected three times to high-pressure (500 bar) homogenization. The mixture is then cooled to 25° C. and phase C is introduced with vigorous stirring. Next, the mixture is again subjected to high-pressure (300 bar) homogenization.

A very fluid, milky, white suspension is obtained, into which the predispersed phase D is introduced.

The white cream obtained, which is smooth and shiny, is capable of preventing or treating skin irritation caused, for example, by household products or solar erythema.

### Example 2

The following test demonstrates the efficacy of the dispersion used according to the invention. This test is carried out in vivo on a panel of 10 individuals sensitive to a solution of sodium lauryl sulphate (irritation initiator). The test includes measuring the change in trans-epidermal water loss and the change in microvascularization using a laser Doppler velocimeter.

The trans-epidermal water loss (TEWL) was measured using an evaporimeter (Servomed) which is a machine that allows the quantitative determination of the evaporation of water from a skin surface (for example from the forearm) according to the method described by Nilsson G. E. “Measurement of water exchange through skin”, in Med. Biol. Eng. Comput. 1977; 15: 208-18. Sensors allow the partial water vapour pressure to be measured at two points located at different distances from the skin surface. It is thus possible to determine the partial water vapour pressure gradient between the two points, and thus the degree of evaporation, in accordance with Fick's law.

The composition of the invention was applied twice a day for four days to the forearm of the individuals. A solution of sodium lauryl sulphate was then applied under an occlusive patch for 24 hours. The TEWL and laser Doppler velocimeter measurements were taken 3 hours after removing the patch. The results are collated in the following table:

| | | Laser Doppler |
|---|---|---|
| | TWEL | velocimeter |
| Naked skin | 41.6 | 401 |
| Composition according to | 34.01 | 226 |
| the invention | | |
| % change | −18% | −43.96% |

These results show that application of the composition according to the invention has a very considerable irritation-preventing effect.

In order to study the curative effect of the composition of the invention, the change in the TEWL and the change in the microvascularization are evaluated 24 hours and 48 hours after removing the patch. The following table gives the results obtained:

| | |
|---|---|
| | Laser Doppler |
| TEWL | velocimeter |

| | 24 h | 48 h | 24 h | 48 h |
|---|---|---|---|---|
| Naked skin | 53.4 | 53 | 206 | 72 |
| Composition according to | 51 | 42.2 | 131 | 36 |
| the invention | | | | |
| % change | −5% | −20% | −36% | −50% |

These results show that the composition according to the invention has a curative effect on inflammation.

This application is based on French Application No. 9710709, filed Aug. 27, 1997, which is incorporated by reference herein in its entirety.

Having now fully described the invention, it will be apparent to one of ordinary skill in the art that many changes and modifications can be made thereto without departing from the spirit or scope of the invention as set forth herein.

What is claimed as new and is desired to be secured by Letters Patent of the United States is:

1. A method for treating skin comprising applying to the skin a composition comprising an aqueous dispersion of oily globules, wherein said globules have a monolamellar or oligolamellar liquid crystal coating, and wherein said method
(a) prevents and/or treats skin disorders associated with inflammation;
(b) treats sensitive skin;
(c) treats skin irritation; and/or
(d) prevents and/or treats solar erythema.

2. The method according to claim 1, wherein the monolamellar or oligolamellar coating is obtained from at least one lipophilic surfactant, from at least one hydrophilic surfactant and from at least one fatty acid, and the dispersion contains a basic agent dissolved in an aqueous phase.

3. The method according to claim 2, wherein the fatty acid contains at least one saturated fatty chain having more than 12 carbon atoms.

4. The method according to claim 3, wherein the saturated fatty chain has from 16 to 22 carbon atoms.

5. The method according to claim 2, wherein the fatty acid is stearic acid.

6. The method according to claim 2, wherein the basic agent is chosen from the group consisting of sodium hydroxide, triethanolamine, lysine and arginine.

7. The method according to claim 1, wherein the monolamellar or oligolamellar coating is obtained from at least one lipophilic surfactant, from at least one hydrophilic surfactant and from at least one ionic amphiphilic lipid which gives the composition a pH ranging from 5.5 to 7.5.

8. The method according to claim 7, wherein the ionic amphiphilic lipid is chosen from the group comprising neutralized anionic lipids, amphoteric lipids and alkylsulphonic derivatives.

9. The method according to claim 7, wherein the ionic amphiphilic lipid is chosen from the mono- and disodium salts of N-stearoylglutamic acid.

10. The method according to claim 7, wherein the lipophilic surfactant and the hydrophilic surfactant each contain at least one saturated fatty chain having more than 12 carbon atoms.

11. The method according to claim 10, wherein the saturated fatty chain has from 16 to 22 carbon atoms.

12. The method according to claim 7, wherein the lipophilic surfactant has hydrophilic-lipophilic balance (HLB) of from 2 to 5.

13. The method according to claim 7, wherein the lipophilic surfactant is sucrose distearate.

14. The method according to claim 7, wherein the hydrophilic surfactant has an HLB of from 8 to 12.

15. The method according to claim 7, wherein the hydrophilic surfactant is sorbitan monostearate polyoxyethylenated with 4 mol of ethylene oxide.

16. The method according to claim 1, wherein the coated oily globules represent from 5 to 50% by weight relative to the total weight of the composition.

17. The method according to claim 16, wherein the coated oily globules represent from 10 to 40%, by weight relative to the total weight of the composition.

18. The method according to claim 1, wherein the composition further comprises a substance chosen from antioxidants, anti-free-radical agents, melanoregulators, tanning accelerators, depigmenting agents, skin-colouring agents, liporegulators, slimming agents, anti-acne agents, antiseborrhoeic agents, anti-aging agents, anti-wrinkle agents, anti-UV agents, keratolytic agents, anti-inflammatory agents, refreshing agents, cicatrizing agents, i4 vascular protection agents, antibacterial agents, antifungal agents, antiperspirants, deodorants, hair conditioners, immunomodulators, nourishing agents, essential oils and fragrances.

19. The method according to claim 1, wherein the composition further comprises one or more cosmetically or dermatologically active, hydrophilic or lipophilic compounds, wherein said compounds are free or encapsulated in vesicles.
